(19) United States
(12) Patent Application Publication
(10) Pub. No.: US 2016/0228702 A1
(43) Pub. Date: Aug. 11, 2016

(54) Neurocranial Electrostimulation Models, Systems, Devices and Methods

(71) Applicant: RESEARCH FOUNDATION OF THE CITY UNIVERSITY OF NEW YORK, New York, NY (US)

(72) Inventors: Rene Kempe, New York, NY (US); Yu Huang, New York, NY (US); Lucas C. Parra, New York, NY (US)

(21) Appl. No.: 15/025,760

(22) PCT Filed: Sep. 30, 2014
(86) PCT No.: PCT/US2014/058350

§ 371 Date: Mar. 29, 2016

Related U.S. Application Data
(63) Continuation-in-part of application No. 13/264,142, filed on Jun. 1, 2012.
(60) Provisional application No. 61/884,620, filed on Sep. 30, 2013.
(60) Provisional application No. 61/168,859, filed on Apr. 13, 2009.

(51) Int. Cl. A61N 1/36; G06F 19/00; A61N 1/04

(52) U.S. Cl. CPC A61N 1/36025; A61N 1/0456; A61N 1/0476; G06F 19/3437

(57) ABSTRACT

Methods, systems, and apparatus, including computer programs encoded on a computer storage medium, for developing transcranial electrical stimulation protocols are disclosed. In one aspect, a method includes obtaining an image of target tissue, assigning to the image tissue electrical conductance values, arranging a plurality of stimulation electrodes around the target tissue, obtaining, from the locations of electrodes and tissue electrical conductances, a forward model of the response of the tissue to applied currents for each electrode and computing an expected tissue response for an arbitrarily sized and shaped electrode defining an electrode area by combining the forward model for each electrode of an array of electrodes covering the defined electrode area.

### CROSS-REFERENCE TO RELATED APPLICATIONS

This application claims the benefit under 35 U.S.C. §119(e) of U.S. Provisional Patent Application No. 61/884,620, entitled “Sampling Pad-Electrodes With High-Definition Arrays for Fast and Accurate Modeling of Transcranial Electric Stimulation, filed Sep. 30, 2013, and is a continuation-in-part application of U.S. patent application Ser. No. 13/264,142, filed Jun. 1, 2012, which claims the benefit under 35 U.S.C. §119(e) of U.S. Patent Application No. 61/168,859, entitled “Neurocranial Electrostimulation Models, systems, Devices and Methods,” filed Apr. 13, 2009, which are all incorporated herein by reference in its entirety.

### BACKGROUND

This specification generally relates to a method and apparatus of electrostimulation of body tissue, including methods of transcranial electrostimulation. For example, plastic changes in brain function can be safely induced in humans by low-intensity electrical stimulation through scalp electrodes. Such electrical stimulation is known as transcranial electrostimulation (TES). These changes can be potentially used for therapeutic or performance enhancing applications. Currently available devices are rudimentary in the sense that they do not target brain modulation and do not apply insights from biophysical studies to functionally target brain function. Moreover, current methods of TES require costly and time consuming computations in order to alter treatment methods.

Transcranial direct current stimulation (TDCS) is a noninvasive neurotechnology, which applies small constant currents (ranging from 0.2 to 2 mA) to the surface of the scalp to achieve neural modulation. TDCS has shown promise in treatment for neurological disorders such as epilepsy, depression, Alzheimer's disease, Parkinson's disease, pain and stroke. It has also been shown to improve cognitive functions, such as memory and learning in healthy individuals. There has also been a recent increase in research on alternating current stimulation (TACS) to affect cognitive function.

Research studies on transcranial electric stimulation, including direct current, often use a computational model to provide guidance on the placing of sponge-electrode pads. However, the expertise and computational resources needed for finite element modeling (FEM) make modeling impractical in a clinical setting.

To estimate current distributions in the brain and to select a proper pad configuration, high-resolution computational models of the human head are often used in clinical research. Current flow models for tDCS are equally applicable for ac stimulation or random noise stimulation so we refer to these collectively as tES.

Intricate folding of the cortical surface, varying skull thickness and distribution of cerebrospinal fluid (CSF) can have a large effect on the current flow inside the head. To include these details it is important to use anatomical head models of at least 1 mm resolution. The necessary spatial resolution for modeling requires complex specialized software and substantial computational resources, making accurate finite element modeling (FEM) modeling often impractical. This is a problem regardless of the specific modeling choice (anisotropy, number of tissues modeled, etc.). Fast modeling solutions that have been proposed to-date do not include CSF or detailed cortical folding. Thus, solutions with the required spatial resolution are not practical for routine use.

### SUMMARY

This specification describes systems and methods relating to electrostimulation of body tissue including methods relating to electrostimulation of transcranial tissue.

In general, one innovative aspect of the subject matter described in this specification can be embodied in methods that include the actions of obtaining a model image of target tissue, developing forward models of the electric field induced in the target tissue for a set of electrodes, and determining from a combination of these forward models the effect of the stimulation corresponding to a specific clinical electrode montage. Other embodiments of this aspect include corresponding systems, apparatus, and computer programs, configured to perform the actions of the methods, encoded on computer storage devices.

An aspect of the subject matter described in this specification can be embodied in methods that include the actions of: obtaining an image of target tissue; assigning to the image tissue electrical conductance values; arranging a plurality of stimulation electrodes around the target tissue; computing, from the locations of electrodes and tissue electrical conductances, forward models of the response of the tissue to applied currents for each electrode; defining desired tissue response; and combining the set of forward models to obtain the desired tissue response for an arbitrary electrode size and shape.

These and other embodiments can each optionally include one or more of the following features. The desired tissue response includes field intensities or currents in a portion of the target tissue and minimal stimulation of a second, different part of the tissue. The desired tissue response includes a change in the volume of tissue activated or a physiological response of the target tissue. The desired tissue response includes a change in the volume of tissue activated or a physiological response of tissue or muscle that is separate and different from the target tissue. The desired tissue response includes strict constraints of maximum allowable currents of field intensities at various tissue locations. The forward model is computed using an finite-element model of the tissue properties. The electrical conductance are non-isotropic and or non-uniform. The parameters altered include changing the voltage, current, activation time, location, sequence or number of electrodes. The combination of the forward models of multiple electrodes is a linear superposition. The linear supposition weights each electrode equally. The forward models are precomputed. The presomputed forward models can be selected from a library of standard head models. The image is derived from a pre-existing image library or a target tissue specific image. The image is derived from a fluoroscopic image, an MRI image, a CT image, or a combination of imaging techniques. The target tissue is transcranial tissue. The electrodes comprise at least two or more electrodes. The electrodes comprise at least 10 or more electrodes. The electrodes comprise at least 100 or more electrodes. The electrodes comprise at least 200 or more electrodes. The electrodes comprise at least 256 or more electrodes. The plurality of electrodes are place around the target tissue based on anatomical landmarks. The plurality of electrodes are placed around the target tissue using the International 10-20 System. The plurality of electrodes are placed around the target tissue in a pattern on the skin, below the skin or within the target tissue. The electrical stimulation applied is a direct current of 0 to 10 mA. The electrical stimulation applied is an alternating current of 0-10 mA and 0 H-1 kHz. The electrical stimulation applied is the same for each electrode in the plurality of electrodes. The electrical stimulation applied is different for each electrode in the plurality of electrodes.

In general, another aspect of the subject matter described in this specification can be embodied in methods that include the actions of accepting an image model of target tissue; obtaining a forward model having a first electrode configuration and first electrical stimulation parameters based on electrical stimulation of the target tissue; accepting electrode configuration changes or electrical stimulation parameter changes resulting in a second electrode configuration or second electrical stimulation parameters; comparing the tissue model with a desired outcome; and providing a confirmation of the model with the desired outcome.

Particular embodiments of the subject matter described in this specification can be implemented so as to realize one or more of the following advantages. Electrode configuration or placement of individual electrodes in the electrode configuration can facilitate: control the volume of (brain) tissue activated by neurocranial electrostimulation; determination of the volume of influence during stimulation; determination of the neurophysiological outcome of stimulation; determination of the clinical or behavioral outcome of stimulation; targeting of a specific brain region such as the cortex, a cortical region, the hippocampus, deep brain structures, axons, axons of passage, pre-frontal cortex, motor region, or sensory regions; treatment of a neurological or psychiatric disease; prevention of tissue damage or prevent cognitive side-effects; determination of the hazards of electrostimulation; accommodation of individual factors, for example to optimize treatment based on patient specific anatomical features; control of stimulation dose; triggering a specific desired response; reduction of stimulation artifact; and creation of a sham stimulation or establishing a control stimulation condition.

Particular embodiments of the subject matter described in this specification can be implemented so as to realize one or more of the following additional advantages. Specific anode/cathode relationships can be established between electrodes, such as neurocranial electrodes, to stimulate the tissue near these electrodes, such as brain tissue near the neurocranial electrodes. Electrodes and other hardware including cranial caps and circuitry can be fabricated based on an optimized model. Clinicians can determine a course of treatment or even if a treatment should proceed based on an optimized model without repeated trial and error stimulation protocols on an actual patient. Electrical stimulation systems for patient treatment can be programmed in accordance with electrode configurations and stimulation parameters identified using the optimized stimulation model. Clinicians can see multiple optimized models without having to run multiple FEM analyses; the cost of stimulation modeling, as reflected in necessary resources (computers), both manual and computational time, supplementary on-site expertise and technical help needed, financial cost, and ability to deploy the optimization in a wide range of environment are reduced. Specifically, the “wait time” for simulation of arbitrary electrode configurations can be done locally by a clinician, for example on a lap-top.

In another aspect of the subject matter described in this specification, a method is performed by data processing apparatus for modeling of transcranial stimulation. The method includes obtaining an image of target tissue, assigning to the image tissue electrical conductance values, arranging a plurality of stimulation electrodes around the target tissue, obtaining, from the locations of electrodes and tissue electrical conductances, a forward model of the response of the tissue to applied currents for each electrode and computing an expected tissue response for an arbitrarily sized and shaped electrode defining an electrode area by combining the forward model for each electrode of an array of electrodes covering the defined electrode area.

In this aspect, each of the plurality of stimulation electrodes is a “small” electrode, which is small enough for the multiple electrodes in the array to not touch each other. In this regard, it is worth noting that better approximations are obtained with increasing numbers of electrodes. However, the surface area should remain largely uncovered to prevent shunting of currents on the surface area of the model, (e.g., ideally not more than 10% of the surface area of the model should be covered), yet the array should be dense enough to densely sample any possible large-pad electrode configuration. The array of electrodes covering the defined electrode area preferably includes at least one high-definition (HD) electrode. The combining of the forward model for each electrode of the array of electrodes covering the defined electrode area involves a linear superposition. Also, the computed expected tissue response preferably includes field intensities or current densities. The forward model can be pre-calculated, wherein the forward model is obtained, for example, from a standard library.

Moreover, in this aspect, pad-electrodes (e.g., 5×5 cm², 5×7 cm²) can be simulated with a subset of high-definition (HD) electrodes (e.g., 4 mm radius) that can be carried out in advance. With this approach a given pad can be quickly simulated as the sum of preexisting solutions computed with high spatial resolution. For each HD electrode in the array simulating the pad a FEM is precomputed one time only. The simulation of the pad is then based on an efficient linear superposition of these individual solutions, which can be readily evaluated for different configurations. Thus, the process of exploring different electrode configurations can be made more efficient by separating it from the computationally demanding FEM modeling.

Conceivably, the FEM modeling of the HD array can be performed once on a set of standard heads, while a large number of clinical researchers and practitioners can then use these solutions to explore and select pad-electrode montages depending of their specific clinical target. In the future, when automated segmentation and modeling becomes more routine, these precomputed solutions could also be generated for individual patients so that practitioners may adjust electrode placement for individual subjects.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an illustration an exemplary image of target tissue.

FIG. 2 is an illustration of an exemplary image of target tissue.

FIG. 3 illustrates an exemplary electrode configuration surrounding target tissue.

FIG. 4 illustrates an exemplary electrode configuration incorporated into a wearable device.

FIG. 5 illustrates a process for determining a forward FEM model of electrical stimulation of target tissue.

FIG. 6 illustrates a process for determining an optimized model of electrical stimulation of target tissue according to the present invention.

FIG. 7 illustrates a process for determining an optimized model of electrical stimulation of target tissue according to the present invention.

FIG. 8 illustrates a process for determining an optimized model of electrical stimulation of target tissue according to the present invention.

FIG. 9 illustrates a two-dimensional schematic of an arrangement of electrodes on a scalp.

FIG. 10a illustrates a head model showing the desired location of an electrode pad.

FIG. 10b illustrates the same head model of FIG. 10a showing an overlay of the desired pad location on an array of electrodes.

FIG. 10c is an enlarged view of the detail area of FIG. 10b

FIG. 11 illustrates electric field distributions on the brain for (a) continuous pad, (b) sampled pad and (c) their difference.

Like reference numbers and designations in the various drawings indicate like elements.

### DETAILED DESCRIPTION

When applying electrostimulation to body tissue, for example neurocranial electrostimulation, decisions must be made regarding the configuration of electrodes and the parameters of the electrical inputs to the electrodes in order to obtain a desired result from the electrostimulation. That is clinicians must determine (1) the positioning of electrodes (how many and where does each electrode go around the targeted tissue), and (2) how much current (or voltage) to apply to each electrode, the duration of the current (or voltage), and the sequence of the activation of electrodes with the specified current (or voltage) for the identified duration. These decisions regarding electrode configuration and stimulation parameters fundamentally affect the outcome of the electrical stimulation on the specific target tissue. One advantage of electrical stimulation is the ability to customize therapy for diseases, anatomical targets, and individuals—but at the same time the many permutations of stimulation configurations (electrode locations, current intensity) make determining an optimal, or even good, configuration challenging. This applies for positioning of electrodes in an implant, but for Neurocranial stimulation using arrays of surface electrode, of potentially high number, there are even more potential permutations. Moreover, electrode configuration and stimulation parameters have typically been determined using multiple trial and error procedures on the patient along with lengthy and costly computer analyses and modeling that are specific to each electrode configuration and stimulation parameter. Computer modeling using forward Finite Element Modeling (“FEM”) analyses have been run for “generic” or standard library tissue images and data, such as generic head models. Forward FEM models have also been run based on individual head data such as measurements and MRI anatomical scans. Such FEM models reduce patient trial and error procedures but involve expansive computer simulations that are specific to each electrode configuration and stimulation parameter associated with each tissue image/model. This is costly and time consuming. Moreover, there are many permutations of potential stimulation configuration resulting in an intractable number of stimulation that would have be run iteratively—in this way an ideal configuration is unlikely to be found. Implementations of the present invention eliminate the need to repeated patient trial and error procedures or multiple matrix or FEM calculations, thereby reducing the time and cost of optimizing electrode configuration and stimulation parameters to achieve a desired outcome from an electrostimulation procedure. Moreover, the implementation in the present invention yields the best electrode configuration and currents with or without additional constraints as practically relevant to the clinical application.

Aspects of the present invention relate to the speed of optimization and the feasibility of optimization of a matrix solution of a particular electrode configuration having a particular stimulation parameter (current or voltage intensity). In one aspect, a series of models are run that calculate the electric field response in the target tissue when one electrode of a plurality of electrodes in an electrode configuration is stimulated at a given current or voltage intensity. In one embodiment, the reference electrode in each case may be an additional fixed electrode or set fixed point in the tissue. In another embodiment, the solutions are from a set of pairs. This model is repeated for each electrode position in the plurality of electrodes in the electrode configuration surrounding the target tissue, (e.g., 100 electrode positions=100 simulations). The same stimulation parameter (e.g., voltage or current intensity) is used for each of run electrode or distinct stimulation parameters are run for each electrode. Regardless, generally only a single stimulation parameter is run for each electrode. Modeling each electrode in the electrode configuration in this manner results in a “forward-model” that is specific to each electrode configuration using a particular voltage or current intensity. In some aspects, the electrode positions can reflect locations on the subject's heads (e.g. 100 electrodes positions around the subjects head). The forward model can then be optimized.

Once this series (e.g. 100 simulations) of models are all solved and the solutions saved as the forward model, predictions can be made as to what electrical fields will develop in the target tissue when any combination of electrodes is activates at any intensity. This is optimizing the forward model and produces the “optimized model.” Moreover, optimizing the forward model according to aspects of the present invention can be done rapidly without the time consuming and costly process of developing a separate matrix or forward model for each desired intensity. Aspects of the invention use pre-solved solutions for each electrode (at one intensity per electrode) to predict what will happen if any combination of the electrodes are activated with any combination of electrode intensities. As such, a clinician wanting to know the outcome of a specific electrode configuration (locations and intensities), can use aspects herein, to immediately predict the electric fields developed in the target tissue in response to the stimulation specified in the optimized model.

Optimization in accordance with aspects of the present invention can also allow determination of the optimal electrode configuration (electrode number, position of each electrode, current at each electrode) based on an outcome specified by the clinician, such as production of a desired electrical field at a desired tissue location. For example the clinician may want to target one part of the brain without affecting neighboring regions of the brain. Rather than the clinician trying the infinite number of combinations possible, the optimization algorithm uses the forward model solved solution series, and automatically calculates the optical electrode configuration.

Developing the Model Image:

Obtaining a two-dimensional or 3-dimensional image of the target tissue, such as a cranial image, and ascertaining tissue properties including electrical characteristics of the tissue such as conductivities, resistivities, permittivities, capacitances, impedances, or applied energies or combinations thereof, allows for development of an “image model” of the target tissue. Image models can be developed from generic library tissue images, fluoroscopy images, MRI images, CT scans or images, other imaging modalities known in the art, or a combination of images. Tissue properties can be developed from library information, testing of the target tissue, or approximated from image data, as described in U.S. Patent Application Pub. No. US 2007/0043268 A1, incorporated herein by reference in its entirety.

A relationship of tissue resistivity to MRI gray scale that can be correlated to tissue types can be expressed by the formula:

R(V)=K(1−v)^{E}+D, where

R=Resistivity;

V=Numeric value of MRI data*;

K=Multiplier value;

E=Exponent; and

D=Density value.

*The V value can be either simple MRI data values or combined values from multiple MRIs or multiple types of MRIs. Exemplary values include K=1600, E=4 and D=65.

Anisotropies/directionalities can be inferred from the anatomy or determined based on the MRI data, or a combination thereof. A direct determination is accomplished by diffusion tensor MRI (DT-MRI, or DTI). The indirect is accomplished by inferring the direction of fibers, specifically nerve fibers, by the general anatomy. DT-MRI data are sometimes called Anisotrophic MRIs.

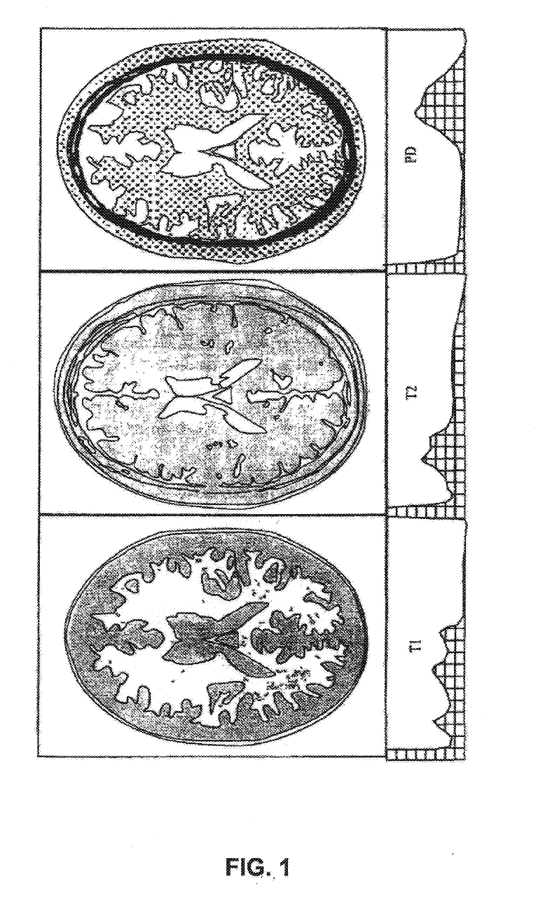

FIG. 1 illustrates MRIs of three different types: T1, T2 and PD. Below each MRI is a gray scale. The gray scale for the T1 MRI appears to resolve three peaks which may correspond to three distinct tissue types having three different resistivities. The gray scale for T2 shows one, or possibly two, peaks, and the gray scale for PD shown one peak at a different resistivity than T2 or T1. By utilizing information from different MRI types, it is possible to enhance gray scale segmentation.

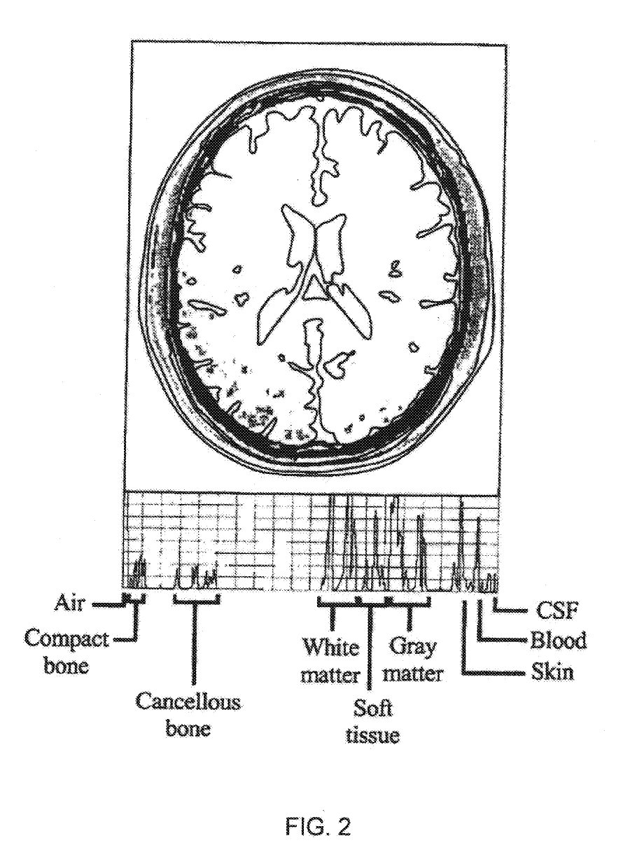

FIG. 2 illustrates a MRI and a plot of resistivities of tissues showing multiple resolved peaks achieved by gray scale differentiation of tissues of different resistivities. The gray scale for the MRI shown in FIG. 1 can resolve multiple peaks corresponding to various tissue types including compact bone, cancellous bone, white matter, soft tissue, gray matter, skin, blood and cerebral spinal fluid. Other resolvable tissues may include cancerous tissue, inflammatory tissue and ischemic tissue, as well as eye fluid. By having enhanced resolution of tissues, it is possible to assign more correctly the vector resistivities or other electrical values to brain or other body tissues, and thereby developing a more accurate image model.

Electrode Configuration:

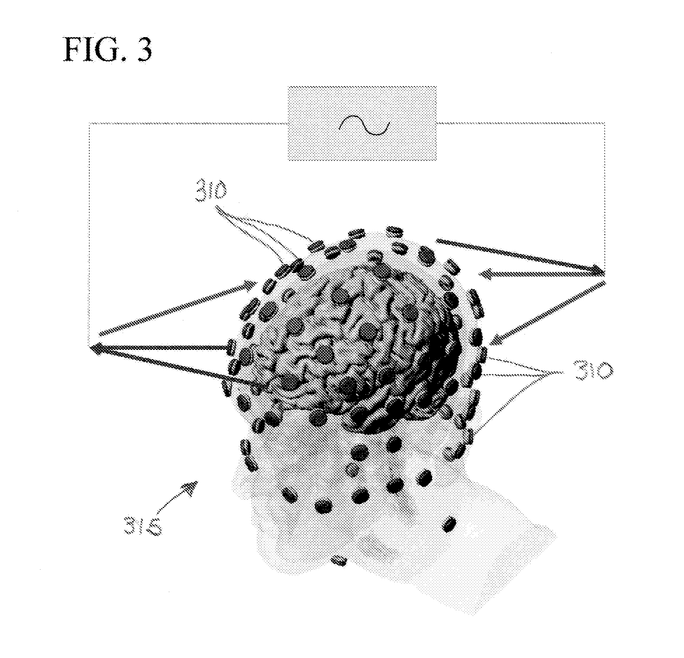

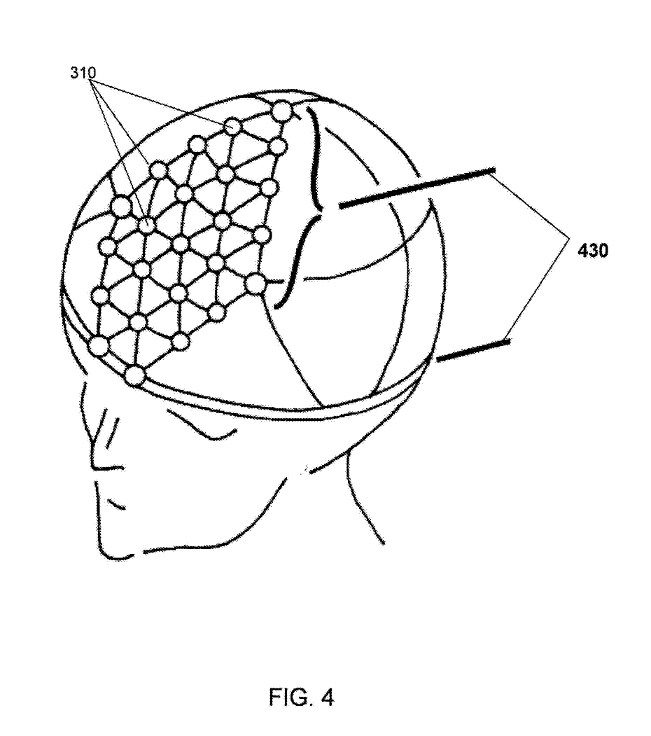

The electrode configuration is placed around the target tissue. The electrode configuration includes two or more electrodes (e.g., three or more electrodes, four or more electrodes, 8 or more electrodes, 10 or more electrodes, 100 or more electrodes, 200 or more electrodes, 256 or more electrodes). The electrodes deliver an electrical stimulation having a desired current or voltage intensity. For examples currents between about 0.1 mA and about 100 mA can be delivered to the target tissue via the electrodes in the electrode configuration. For example, voltages of between about 0.1V and about 100V can be used. Not all electrodes in the configuration need to be active. That is, in certain embodiments, some electrodes may have zero current, 0 volts while others may deliver the desired current or voltage intensity. FIG. 3 illustrates an exemplary electrode configuration around target cranial tissue, where electrodes 310 are placed around the subjects head 315. Electrodes 310 can be placed randomly or according to established systems, such as the International 10-20 system. Electrodes can be placed around the target tissue based on anatomical landmarks. Electrodes 310 can be placed on the surface of the skin, under the skin, or within the target tissue. Electrodes 310 can be incorporated into a wearable device, such as the skull cap 430 illustrated in FIG. 4.

Developing the Forward Model

Development of the forward model involves determining the electric field response in the target tissue for each electrode in the electrode configuration at a specified stimulation parameter such as current or voltage intensity. The electric field response of the target tissue can be described as the volume of tissue activated in response to the electrical stimulation.

There are different ways to determine the “volume of tissue activated”. At the most basic level this includes induced brain voltage (rarely used), electric fields (same as current density), and electric field derivative (same as activating function). On a more complex level, the calculations of induced voltage/field in the brain are than passed through another set of equations that more accurately predict brain activation. These second stage filters can to include 1) at the simplest a threshold (e.g. electric field greater than x is full activation, less than x is no activation); 2) the electric field may be broken down into direction where the direction themselves are based on the cortical geometry (for example the electric field perpendicular to the cortical surface) or sub-cortical anatomy (for example direction of axons); 3) the effects of electric fields on neurons may be directly computational determined (e.g. a (detailed) model so a cortical neuron). On a yet more complex level, system level and control theory analysis may be used to predict effects of cognition and behavior. Any combination of this “volume of tissue activated” solutions can form a “prior-set” of solutions wherein a set of solutions for each electrode in the electrode configuration forms the forward model.

The volume of tissue activated can be determined based on a spheres model, an individualized spheres model, and anatomical model based on gross measurements, and anatomical model based on individual images such as MRI. The above “volume of tissue activated” can be determined for a sub-set of potential stimulation configurations. Where a stimulation configuration used a set of one or more electrodes, in a specific arrangement, and a certain level of current delivered to each electrode. The number of independent configurations are tested and the “volume of tissue activated” is determined for each electrode configuration. This forms a pre-determined set of solutions. There is a time cost associated with determined the volume of tissue activated.

Finite Element Modeling (FEM) as a method for determining the volume of tissue activated can be used with the image model and the specified electrode configuration to produce the forward model. FEM techniques are described in U.S. Patent Application Pub. Nos. US 2007/0043268 A1 and US 2006/0017749 A1, both of which are incorporated herein by reference in their entirety. FEM analysis may be done using any number of commercial computer programs, such as FEMLAB by Comsol Pty. Ltd. of Burlington, Mass.

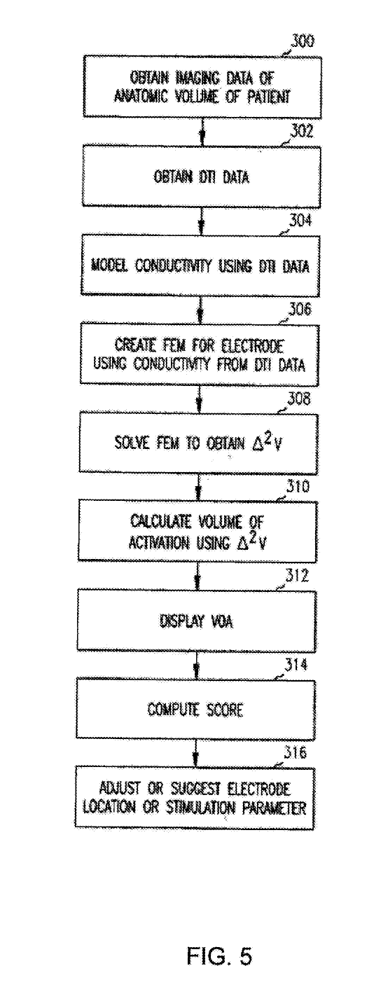

FIG. 5 is a flow chart illustrating generally one example of a method of using a model to calculate a volume of activation, as discussed above. Portions of the method may be embodied in any machine-accessible medium carrying instructions for executing acts included in the method. Such a method applies to deep brain stimulation (DBS) or any other electrical tissue stimulation. At 300, imaging data of an anatomic volume of a patient is obtained. In one example, this includes obtaining imaging data of a patient's brain using an imaging modality, such as computed tomography (CT) or magnetic resonance (MR) imaging modalities, for example, or another suitable imaging modality. The anatomic volume need not be all or part of the patient's brain, but could be all or part of any other anatomic structure.

At 302, in one example, diffusion tensor imaging (DTI) data is obtained (this may occur at 300, such as where a DTI MR imaging modality is used at 300). In one example, the DTI data is obtained from the same patient being analyzed. Alternatively, “atlas” or library DTI data is obtained from at least one other patient. If atlas DTI data from another patient is used, it is typically spatially scaled to correspond to the anatomic size and shape of the patient being analyzed. In one example, the atlas DTI data is based on a composite from more than one other patient. The composite atlas DTI data typically spatially scales DTI data from the different patients before combining into the composite DTI atlas. The atlas DTI data avoids the need to obtain DTI data from the particular patient being analyzed. This is useful, for example, when a non-DTI imaging modality is used at 300.

At 304, a tissue conductivity model, or image model as discussed above, is created for all or part of the anatomic volume. The tissue conductivity model is typically a non-uniform spatial distribution. Such a model more accurately represents inhomogeneous and anisotropic characteristics of the tissue anatomy. For example, the conductivity of brain tissue varies from one brain region to another. Moreover, conductivity of the nervous system is preferential to a particular direction that is also dependent on the particular location in the brain. In one example, a non-uniform tissue conductivity model is created by transforming the DTI data into conductivity data, such as by using linear transform techniques known in the art.

It should be noted that it is not required to obtain non-uniform tissue conductivity data using DTI. There exist several alternatives to using DTI based approximations for the anisotropic and inhomogeneous tissue properties for the patient specific finite element volume conductor model. One example technique would be a simple designation of a white matter and a grey matter conductivity tensor, as discussed above. These two universal conductivity tensors could then be applied to the nodes of the FEM mesh using co-registration with the anatomical MRI. In this manner, the individual voxels of the MRI data are designated as either white matter or grey matter using post-processing image analysis. Then, each such voxel is assigned a conductivity dependent on whether it was classified as white matter or grey matter, which white matter voxels having a different conductivity value than grey matter voxels. A second example technique would define individual conductivity tensors for designated brain regions (e.g., nuclei, sub-nuclei, fiber tracts, etc.). This method would allow for a more detailed representation of the tissue electrical properties than the first example technique. The conductivity tensor of each designated brain region is defined, in one example, using explicit experimental tissue impedance results and anatomical information provided by a human brain atlas. In this technique, the anatomical MRI is sub-divided into different designated brain regions on a voxel-by-voxel basis using post-processing image analysis. The appropriate conductivity tensors for each designated brain region is then co-registered with the nodes of the FEM mesh.

At 306, a finite element model (FEM) is created using the conductivity data obtained at 304. In one example, the FEM model uses a default boundary condition that is appropriate for a typical electrode contact morphology. However, in another example, the FEM model includes an electrode-specific boundary condition that is tailored to the morphology of a particular electrode contact or contacts to be used in the DBS or other procedure. The FEM model provides for non-uniform conductivity in the tissue, such as by using a DTI-derived other conductivity value at each node in the FEM mesh. The FEM model may include aspects that are not obtained from the DTI-derived data. In one such example, the FEM mesh models a thin encapsulation sheath about the electrode lead body, as discussed above, which is not derived from the DTI data.

At 308, in one example, the FEM is solved for the electric potential distribution or the second difference (.DELTA²V) of the electric potential distribution, such as by using FEM solver software. In one example, the FEM is solved for a normalized stimulation amplitude of 1V or 0.1 mA. In another example, for a different electric stimulation amplitude, the resulting electric potential distribution (or second difference of the electric potential distribution) is multiplied by a scale ratio of the different electric stimulation amplitude to the normalized electric stimulation amplitude.

Scoring of Solutions Based on VOA

In one embodiment as outlined below, solutions are optimized to reduce the error between a desired electric field distribution and the electric field distribution achieved by applying the optimized current to electrodes. In other embodiments additional steps are taken to SCORE solutions based on additional analysis including volume of activation (VOA). Scoring may be integrated into the optimization process as described below, or used as an additional processing, data display, and user interface step. The prior art solved and scored each electrode configuration and current individually and iteratively without optimization at tremendous computational and time cost.

At 310, a volume of tissue activation (VOA) or other volume of influence is calculated, in one example, using the second difference of the electric potential distribution. The VOA represents the region in which any neurons therein are expected to typically be activated, that is, they are expected to generate propagating action potentials at the stimulus frequency in response to the electrical stimulation delivered at the stimulation electrode contact. Conversely, neurons outside the VOA are expected to typically remain inactivated in response to the electrical stimulation. Neurons in the volume of influence are expected to be modulated (neuromodulatoin) in a manner related to the strength of regional electric field or some function there-of. In one example, a particular threshold value of the second difference of the electric potential distribution defines the boundary surface of the VOA.

As discussed above, the particular threshold value defining the boundary of the VOA is determined as follows. First, model neuronal elements are positioned relative to the electrode using known neuroanatomical information about specific fiber pathways and nuclei of interest near the electrode. These generalized positions of the model neuronal elements are then refined, such as by using explicit “patient-specific” information provided in the DTI or anatomical MR imaging data. For example, the DTI imaging data describes the inhomogeneous and anisotropic tissue properties near the electrode. In this example, such DTI imaging data is used to explicitly define one or more axonal trajectories, if needed, or to help define nuclear boundaries specified in the anatomical MRI.

A model of these neurons is then created. In one example, the neurons are modeled using an axon model, which is a simplified form of a neuron model. An example of an axon model is described in Cameron C. McIntyre et al., “Modeling the Excitability of Mammalian Nerve Fibers Influence of Afterpotentials on the Recovery Cycle,” J. Neurophysiology, Vol. 87, February 2002, pp. 995-1006, which is incorporated by reference herein in its entirety, including its disclosure of axon models. In another example, a more generalized neuronal model is used, an example of which is described in Cameron C. McIntyre et al., “Cellular Effects of Deep Brain Stimulation: Model-Based Analysis of Activation and Inhibition,” J. Neurophysiology, Vol. 91, April 2004, pp. 1457-1469, which is incorporated by reference herein in its entirety, including its disclosure of neuronal models. The neuron model describes how the neurons will respond to an applied electric field, that is, whether the neuron will fire and whether the neurons will generate a propagating action potential.

In one example, using this neuron model to simulate how the neurons (located as determined from the DTI-derived conductivity data, in one example) behave, the threshold value of the second difference of electric field that will result in such propagating action potentials is calculated. The stimulating influence of the electric field is applied to the model neurons to define a threshold value. This threshold value is then used to define the boundary of the VOA in the non-uniform conductivity tissue, as discussed above.

It should be noted that calculation of explicit threshold criteria for each patient is not required. For example, in a more generalized situation, threshold criteria will have already been determined using the detailed neuron models under a wide variety of different stimulation conditions. Once these threshold criteria have been determined, they need not be re-determined for each subsequent patient.

It should also be noted that using a threshold criteria upon the second difference of the potential distribution in the tissue medium is a simplified technique for quickly determining a VOA or other volume of influence. The intermediate step of using the second difference of the potential distribution is not required. In an alternate example, the FEM model is directly coupled to a detailed neuron model, such as a multi-compartment neuron model that is oriented and positioned in the FEM model to represent at least one actual nerve pathway in the anatomic volume.

At 312, the calculated VOA region is displayed, such as on a computer monitor. In one example, the VOA is displayed superimposed on the displayed imaging data or a volumetric representation derived from such imaging data. In another example, an anatomic boundary or other representation of an anatomic structure is superimposed on the VOA and imaging data or the like. The anatomic boundary data is typically obtained from an atlas of brain anatomy data, which can be scaled for the particular patient, as discussed above. Alternatively, the anatomic representation is extracted from the imaging data for the patient being analyzed. In one example, the anatomic representation is a line depicting one or more boundaries between particular nucleus structures or other regions of the brain, such as the STN, IC, or ZI illustrated above in FIG. 1B.

In any case, by viewing a representation emphasizing one or more brain regions displayed together with the VOA, the user can then determine whether a particular anatomic region falls within or outside of the modeled VOA. The user may want a particular anatomic region to be affected by the DBS, in which case that region should fall within the modeled VOA. Alternatively, the user may want a particular region to be unaffected by the DBS, such as to avoid certain unwanted DBS stimulation side effects, as discussed above. This evaluation of whether the VOA is properly located can alternatively be performed by, or assisted by, a computer algorithm.

For example, the computer algorithm can evaluate various VOA's against either or both of the following input criteria: (a) one or more regions in which activation is desired; or (b) one or more regions in which activation should be avoided. In one example, at 314, the computer algorithm creates a score of how such candidate VOAs map against desired and undesired regions. In one example, the score is computed by counting how many VOA voxels map to the one or more regions in which activation is desired, then counting how many VOA voxels map to the one or more regions in which activation is undesired, and subtracting the second quantity from the first to yield the score. In another example, these two quantities may be weighted differently such as, for example, when avoiding activation of certain regions is more important than obtaining activation of other regions (or vice-versa). In yet another example, these two quantities may be used as separate scores.

At 316, the score can be displayed to the user to help the user select a particular VOA (represented by a particular electrode location and parameter settings). Alternatively, the algorithm can also automatically select the target electrode location and parameter settings that provide the best score for the given input criteria.

In one example, the VOA is displayed on a computer display monitor of an image-guided surgical (IGS) workstation, such as the StealthStation® from the Surgical Navigation Technologies, Inc. (SNT) subsidiary of Medtronic, Inc., for example. The VOA can be displayed on the IGS workstation monitor with at least one of the imaging data representing the anatomic volume, the target electrode location, a burr hole or other anatomic entry point, a trajectory between the anatomic entry point and the target electrode location, or an actual electrode location.

In one IGS workstation example, the displayed VOA corresponds to a target electrode location.

After the electrode is positioned at the target location, there remains the challenging task of adjusting the DBS stimulation parameters (e.g., the particular electrode contact(s) of a plurality of electrode contacts disposed on the same DBS leadwire, pulse amplitude, pulsewidth, electrode “polarity” (i.e., monopolar or bipolar electrode return path), electrode pulse polarity (i.e., positive or negative), frequency, etc.). In one example, the IGS workstation or a DBS pulse generator programmer includes the above-described VOA methods to assist the user in selecting an appropriate combination of DBS stimulation parameters, such as by using the scoring techniques discussed above. In essence, for each electrode position, a forward model can be calculated. As discussed above, this is time consuming and costly.

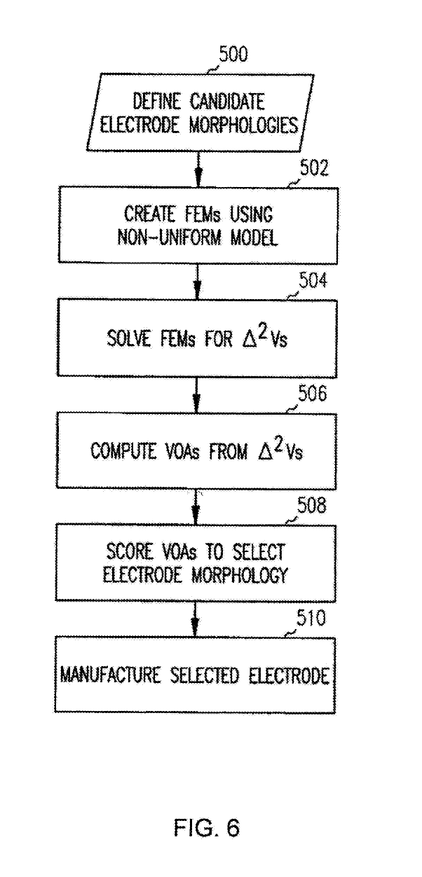

FIG. 6 is a flow chart illustrating generally one example of a method of using an FEM model to calculate a volume of activation, as discussed above, and using the volume of tissue activation to select a particular electrode configuration. Portions of the method may be embodied in any machine-accessible medium carrying instructions for executing acts included in the method. Such a method applies to selecting an electrode configuration for deep brain stimulation (DBS) or for any other electrical tissue stimulation. At 500, a set of N candidate electrodes are defined, where N is an integer greater than 1. Defining the candidate electrode configurations typically includes providing information about the size, shape, or arrangement of electrode contacts on a leadwire. Such information is typically in a form in which it can be used as input to a finite element model (FEM). At 502, a FEM is created for each candidate electrode position. The FEMs typically use non-uniform conductivity model of a desired region of interest. At 504, each FEM is solved for a second difference in the electric potential distribution. At 506, a volume of activation (VOA) is computed for each candidate electrode morphology from its corresponding second difference in the electric potential distribution. The boundary of the VOA is typically obtained from a threshold value that is based on a neuron or axon model, as discussed above. At 508, the VOAs are scored, as discussed above, or otherwise evaluated to select one or more electrode locations that exhibit a desired VOA, or a VOA that is deemed more desirable than the VOA of one or more other electrode morphologies. At 510, at least one electrode is manufactured using the selected at least one electrode morphology.

Optimizing the Forward Model:

The above image collection and modeling techniques require time intensive and costly computations to determine the volume of tissue activated for a particular electrode configuration using specific stimulation parameters. Moreover, changes to electrode positions and or changes to stimulation parameters, such as current or voltage intensities, require recomputation to verify the new volume of tissue activated. For example, to solve and score a multitude of solution as described above is very costly computationally and may not result in an optimal configuration.

It has been found that changes to the forward model, and particularly changes to current or voltage intensities can be optimized or predicted using linear approximations. This avoids the need for re-running costly computational forward models and greatly enhances the speed to which electrical stimulation procedures can be modeled, outcomes predicted, and treatments prescribed.

In an aspect, the governing equation for the optimization process is given by the simple linear relationship:

E=MI (1)

where:

E is a vector that represents the field magnitudes at multiple locations in the tissue.

I is a vector that represents the currents at multiple electrodes.

M is a matrix that linearly relates the currents I to the fields E and is called a “forward model”.

The goal of optimization is to choose an optimal current vector I* with a net-zero to current such that desired field magnitudes E* are achieved, while maintaining maximum current limits:

I*=argmin∥E*−MI²∥ subject to constraint I<Iₘₐₓ and sum(I)=0 (2)

Other constraints such as “minimum number of non-zero currents”, “maximum allowable field intensities” or “maximum allowable current densities” can be incorporated into this approach using additional optimization criteria (such as an L1-norm penalty term) or linear boundary constraints.

The forward model M can be computed from an 3D distribution of electrical conductances and the locations of the electrodes and locations for which the field is to be computed. The values in matrix M are the solution to the quasi-static Laplace equation (simplification of Maxwell equations) under Dirichlet boundary conditions which govern the relationship between static currents I, ie. the boundary conditions, and the resulting electric fields E, i.e. the solutions, in a purely resistive material. Each column in matrix M represents the solutions for a unit current in a given electrode pair (all other electrode carrying no current). Each row in matrix M represents a different location in the tissue. If N electrodes are used and a single electrode is used as a common reference for all pairs, then matrix M has N−1 columns. The number of rows of matrix M scales with the number of 3D locations in the tissue for which one would like to specify field magnitudes. To be more specific, there is one such matrix for each field orientation: Mₓ for lateral-direction field Eₓ, M_{y} for depth-direction field E_{y} and M_{z} for vertical-direction field E_{z}:

M=[Mₓ^{T}, M_{y}^{T}, M_{y}^{T}]^{T}, and E=[Eₓ^{t}, E_{y}^{T}, E_{y}^{T}]^{T}. Thus the number of rows is precisely 3 times the number of tissue locations of interest.

The 3D current distribution can be obtained, for instance, from a segmentation of a 3D image of the tissue such as MRI, CT, DTI, etc. Such a segmentation can be reformulated in a finite-element model (FEM), which is then used to provide an efficient numerical solution to the Laplace equation. Commercial software such as Abacus or Comsole is available to perform these numerical FEM computation.

In an example, the optimization problem given in equation (2) can be formulated on the basis of the linear relationship (1) and that matrix M does not have to be recomputed using computationally intensive numerical approaches for every possible choice of I. Thus the optimization can be solved strictly using efficient least-squares optimization procedures and do not require an expensive trial-and-error search for I.

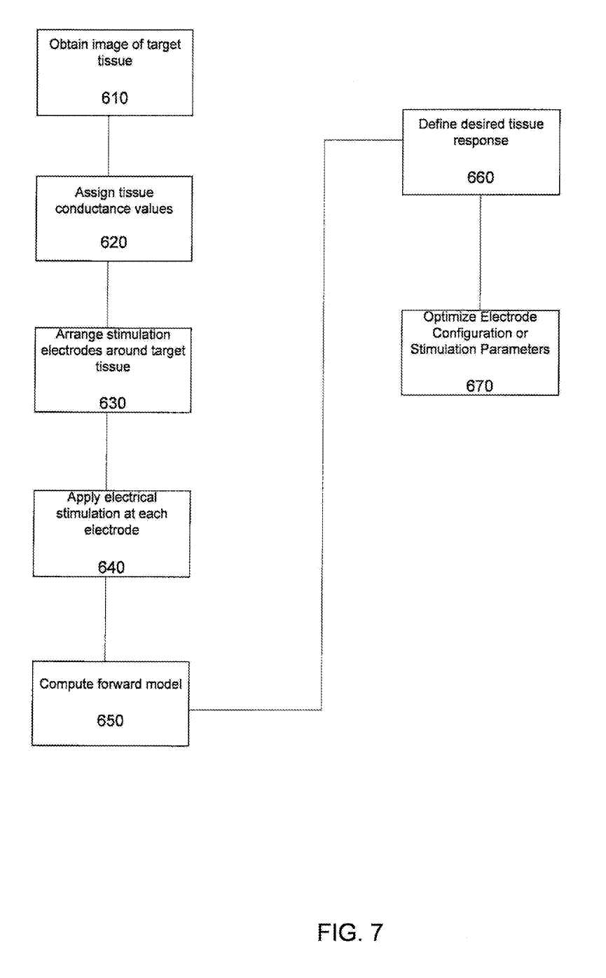

FIG. 7 is a flow chart illustrating generally one example of optimizing a model to determine or predict changes to a stimulation procedure, as discussed above. Portions of the method may be embodied in any machine accessible medium carrying instructions for executing acts included in the method. Such a method applies to electrical stimulation of bodily tissue, including transcranial tissue. At 610, an image of target tissue is obtained, as discussed above. The image can be obtained from generic library tissue images, fluoroscopy images, MRI images, CT scans or images, or a combination of images. The target tissue can be any bodily tissue including transcranial tissue.

At 620 conductance values are determined for the target tissue. Conductance values can be a uniform or a non-uniform spatial distribution. Conductance values can be determined from pre-existing data of generic tissue images, target tissue specific data derived from testing, or determined from grey scale MRI images of the target data as described above.

At 630, electrodes are configured about the target tissue. For example, electrodes are distributed around a patient's head according to the International 10-20 System. The number of electrodes can range from 2 or more, 3 or more, 10 or more, 100 or more 200 or more, and 256 or more. A current and voltage intensity is determined. The applied current can be in the range of about 0.1 mA to about 100 mA. The voltage can be in the range of about 1V to about 100V. In aspects, the same current or voltage intensity is used for each electrode in the electrode configuration.

At 640, electrical stimulation is applied to each electrode, using the same current and voltage intensity. For each electrode stimulation, the volume of tissue activated is calculated. Cataloguing each volume of tissue activated for each electrode stimulation results in the development of the forward model at 650.

At 660 a desired tissue response is defined. For example, a particular volume of tissue activated may be desired. Other examples include maximizing one are of tissue activation while minimizing other areas of tissue activation. A particular physiological response may be desired.

At 670, the forward model is optimized, as described above, to predict the electrode configuration and current or voltage intensities required to produce the desired tissue response.

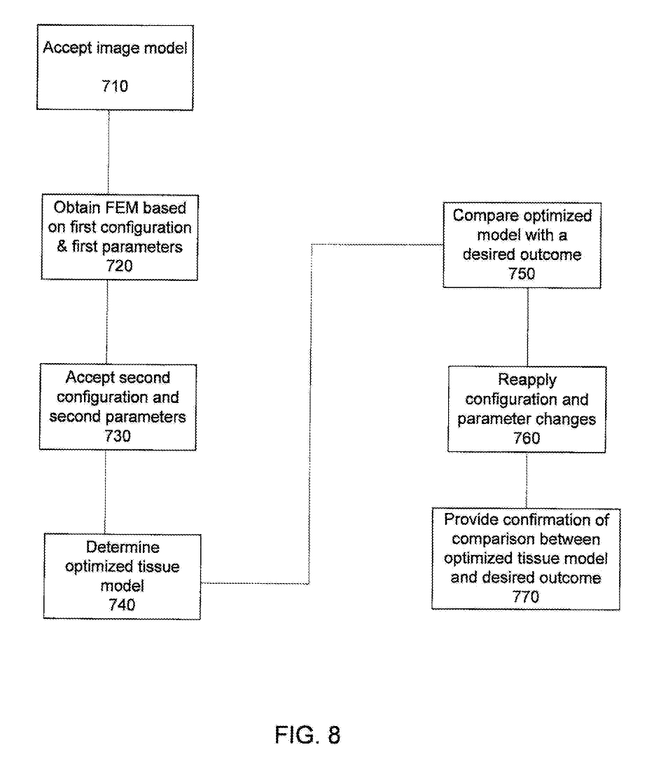

FIG. 8 is a flow chart illustrating generally one example of optimizing a model to determine or predict changes to a stimulation procedure, as discussed above. Portions of the method may be embodied in any machine accessible medium carrying instructions for executing acts included in the method. Such a method applies to electrical stimulation of bodily tissue, including transcranial tissue.

At 710, an image model, as described above is accepted. The image model includes a 2-D or 3-D image of the target tissue along with certain electrical characteristics of the target tissue. For example, the image model can be of transcranial tissue and can include tissue conductance values. It will be noted that the image model need not be developed locally but can be down loaded from a file. The image model can be generic image model or a patient specific image model.

At 720 Finite Element Model (FEM) of the image model is obtained. The FEM is based on a particular electrode configuration, such as electrodes arranged according to the International 10-20 System. The number of electrodes can range from 2 or more, 3 or more, 10 or more, 100 or more 200 or more, and 256 or more. The FEM is also based on a particular stimulation parameter (e.g., current and voltage intensities). A current and voltage intensity is determined. The applied current can be in the range of about 0.1 mA to about 100 mA. The voltage can be in the range of about 1 V to about 100V. In aspects, the same current or voltage intensity is used for each electrode in the electrode configuration. It will be noted that the FEM does not need to be developed locally but can be downloaded from a file.

At 730 a second electrode configuration (e.g., movement of one or more electrodes in the first electrode configuration) and/or a second stimulation parameter (e.g., a change in the current or voltage intensity from the first stimulation parameter) is accepted.

At 740 the FEM is optimized using the optimization formula described above to determine the tissue response from the changes in electrode configuration or stimulation parameters. In an example, a desired tissue response is determined and the electrode configuration and stimulation parameter predicted utilizing the optimization formula described above. At 750 the optimized model can be compared with a desired outcome or physiological response. In an example, the optimized model can be compared against certain constraints, such as maximum voltages or currents at one electrode or all electrodes, or maximum electrical fields at any tissue location.

At 760, the electrode configuration and stimulation parameters can be adjusted based on the optimized model and the desired outcome or known constraints. The desired outcome can be adjusted based on the electrode configuration and stimulation parameters used to optimize the model.

At 770, confirmation of the optimized model and the desired outcome, along with the electrode configurations and stimulation parameters can be provided to a user via a user interface, a print-out or other means known in the art.

### Additional Embodiments

Aspects of the present invention use a matrix set of solutions to determine the “volume of tissue activated” or “region of influence” for a separated electrode configuration (one in which the volume of tissue activated has not been determined)—this is a new electrode configuration of interest. This is done by combining the set of solutions in a manner that minimizes error. The solutions may, for example, be each weighted linearly such that there is a weight associated with each solution, and the each solution multiplied by this weight added. For example, a series of solutions of electric fields in the brain. The weight may be determined such that the electrical current injection by the summation of each solution multiplied by its respective weight equals the electrical current injection by the new electrode configuration of interest. The weight may be determined by minimizing the error between the combined electrical current injected by the set of solution multiplied by the weights and the electrical current injection by the configuration of interest. The weight may be determined by other means including random selection and testing or based in a linear summation or prior knowledge about the electrode configurations. The time cost of determining these weights, and then by multiple each prior solution bits respective weights, and then addition of this new multiplied set of solutions, is less than the time cost of running a forward model (e.g., a finite element model) of the new configuration. The computational cost of determining these weights, then multiplying each prior solution by its respective weights, and then addition of this new multiplied set of solutions, is less than the computational cost of running a forward model of the new configuration. The complexity cost of determining these weights, then multiplying each prior solution bits respective weights, and then addition of this new multiplied set of solutions, is less than the complexity cost of running a forward model of the new configuration.

In another aspect, the weights applied to each prior-solution of volume of tissue activation are determined by an optimization algorithm. The optimization algorithm determines the optimal weight that may be applied to the prior-set of solutions to achieve a desired outcome. The optimization algorithm achieves this at a time and computational cost that is less than trying every possible electrode configuration. The optimization algorithm compares the set of prior solutions (e.g., prior volume of tissue activation) with the solution corresponding to the desired clinical outcome (e.g., new volume of tissue activation) and then determines the weight necessary to make these as “close as possible.” The criteria for “as close as possible” are determined by pre-set conditions of the operator and can include minimizing the difference between the clinically desired “volume of tissue activation” and the volume of tissue activation by a configuration determined by the algorithm.

In one implementation the following steps are taken: The electric fields generated (forward model FEM) from monopole stimulation with a single electrode with 1 mA (or 1V) is calculated using a head model. This prior-solution is done for 128 electrode positions. The return electrode is at a fixed position for all electrode stimulations. This results in a set of 128 prior-solutions of electric fields in the head. This can be done “off-line” with a higher time and computational cost. The user interface allows a clinician to select to activate any combination of these 128 electrodes with any level of current (or voltage) at the same time. This is done by the clinician indicating how much current X to apply at each of the 128 electrodes. The position of each of these electrodes corresponds to the position of electrodes in the prior-solution. The current at each electrode does not have to be the same and can be zero. (On-line) the electric field generated by the clinician selected configuration is determined as follows:

For each electrode selected by the clinician a level of current X is specified. The prior solution (for 1 mA or 1 V) associated with each electrode is multiplied by X. This new set of solutions is then added together. The time and computational cost associated with this operation is minimized. The results of this operation are displayed to the clinicians. The results of this operation can determine the conditions of stimulation.

In another implementation the following steps are taken: The electric fields are generated (forward head model FEM) from monopole stimulation with a single electrode with 1 mA (or 1V) and calculated using a head model. This prior-solution is done for 128 electrode positions. The return electrode is at a fixed position for all electrode stimulations. This results in a set of 128 prior-solutions of electric fields in the head. This can be done “off-line” with a higher time and computational cost. The user interface allows a clinician to select a desired electric field distribution in the head. How much current to apply to each electrode to achieve this desired electric field distribution in the head is then calculated. This is done by comparing the set of prior-solutions with the desired electric field distribution and then determining how much current to apply to each electrode to minimize the difference between the desired electric field distribution and the electric field distribution induced by applied said current to the electrodes. The results of this operation are displayed to the clinicians. The results of this operation can determine the conditions of stimulation.

In another example, software calculates the stimulation efficacy and target specificity for a given stimulation protocol. This software includes a 3-D representation of the head impedance including the scalp, skull, CSF, and brain compartments. The 3-D representation requires manually-assisted segmentation of anatomical MRI scans. The user (clinician) can place two or more electrodes on the segmented scalp surface and simulate brain modulation and iteratively “explore” high density transcranial electrostimulation configurations. In prior-art applications, for each configuration change, the predicted brain modulation must be “re-solved”, which takes time. In addition, placing the electrodes and determination of the current flows has been an ad-hock process. The optimal configuration may ultimately depend on an integration of patient, disease, and clinical experience related factors, and it is thus useful for a clinician to be able to intuitively and rapidly evaluate a range of configurations.

The high spatial specificity provided by in aspects of the present invention offers the opportunity to design a stimulation protocol that is tailored to the individual subject and the desired target location. Specifically, the task for the targeting methodologies disclosed herein is to determine the ideal electrode locations and to compute the required current flows through each of these electrodes; moreover the targeting processes should be accessible to a clinician using a PC, desktop computer, or portable computing device. Three technical challenges associated with the targeting processes disclosed herein include: (1) Automatic patient specific MRI segmentation; (2) Acceleration of the computational time for predicting brain modulation in response to a given stimulation configuration (3) Computation of the optimal electrode locations and currents for a user selected brain target.

Forward Model

High resolution (gyri/sulci precise) MRI derived finite element (FE) human head models were generated by segmenting grey matter, white matter, CSF, skull, muscle, fatty tissue, eyes, blood vessels, scalp etc. Each model may comprise >10 million elements with >15 million degrees of freedom. The induced cortical electric field/current density values are calculated and used to predict regions of brain modulation.

Subject Specific

To obtain a subject specific prediction of current flows existing software packages were used to automatically segment a given subject's anatomical MRI. As significant effort by the research community has already generated a number of publicly available segmentation packages that focus on MRI segmentation (Shattuck & Leahy, 2002, Dogdas 2005, see brainsuite.usc.edu). These are adapted to integrate with the commercial FEM solver such as SIMPLEWARE (SIMPLEWARE LTD. to import MRI scans, segmentation, creating FE mesh, and COMSOL INC. for FEM computation). The result of this integration work was a software package that takes an anatomical MRI and computes the required forward models for each electrode location.

Variability in anatomy is well characterized by head size, gender, and age. Therefore, a set of standard forward models can be computed and provided as part of the real-time clinician interface to cover a range of subjects to be selected according to these criteria. If a subject-specific MRI is available the corresponding forward-model can be computed remotely using high-end computers.

In another example, computationally costly segmentation and FEM solving is run on a patient specific basis, while physician targeting software can operate on real-time by virtue of using pre-solved FEM solutions.

Another example relates to real-time optimization of stimulation protocols. It has been found that current flows within the brain are a linear superposition of the currents generated by each electrode. Based on this observation the optimization problem can be formulated as a constrained least-squares problem. Least squares aims to minimize the square difference of a desired current distribution (specified by the clinician) with the achievable current distribution as predicted by the FEM. The method is computationally efficient as the predicted current distribution can be formulated as a linear combination of the “forward model” that has been previously computed for each electrode. The forward model computation using the FEM is computationally expensive and can be performed remotely (along with segmentation).

Standard electrode locations to facilitate proper electrode placement (10/20 system conventionally used in EEG). While this is not typically required, one can increase specificity by confirming electrode locations with conventional 3D electrode-localization hardware (e.g. Fastrak, Patriot, see cortechsolutions.com). The constraints in the optimization procedure implement the requirements that currents at each electrode do not exceed some threshold value (corresponding to skin irritation and perception limits) as well as limits on maximum current flows within the tissue (safety limits). Analogous optimization problems are adapted with sensor arrays in acoustics (Parra, 2006). The computationally expensive step of computing the FEM is performed in advance for a set of standard electrode locations (, while the optimization of the electrode currents to target a specific cortical area is performed in real-time. A GUI allows the physician to define the target location and inspect the expected optimal current distribution.

Targeting Software

In another example, targeting software calculates the stimulation efficacy and target specificity for a given stimulation protocol. This software includes a 3-D representation of the head impedance including the all significant anatomical compartments. In the system the 3-D representation requires manually-assisted segmentation of anatomical MRI scans. The user (experimenter) can place two or more electrodes on the segmented scalp surface and predict resulting current distributions on the brain. In the current system, for each configuration change, the predicted current distribution in the brain must be recomputed, which takes time. In addition, placing the electrodes and deciding on the adequacy of resulting current flows is currently an ad-hoc process. The optimal configuration may ultimately depend on an integration of various factors, including patient brain anatomy, desired target area, safety and efficacy considerations. This software allows the experimenter to intuitively and rapidly evaluate a range of electrode configurations.

The high spatial specificity provided by HD-TES offers the opportunity to design a stimulation protocol that is tailored to the individual subject and the desired target location. Specifically, the task for the HD-TES targeting software is to determine the ideal electrode locations and to compute the required current flows through each of these electrodes; moreover the targeting software should be accessible to an experimenter using a conventional PC. There are three technical challenges related to this task: (1) Automatic patient specific MRI segmentation; (2) Acceleration of the computational time for predicting brain modulation in response to a given stimulation configuration (3) Computation of the optimal electrode locations and currents for a user selected brain target. These challenges can be addressed with two software modules, one for computing the head model of the current flows based on anatomical MRI, and the second for optimizing the applied currents and electrode locations.

Subject Specific Forward Model.

To obtain a subject specific prediction of current flows, existing software packages can be used to automatically segment a given subject's anatomical MRI. As significant effort by the research community has already generated a number of publicly available segmentation packages that focus on MRI segmentation (Shattuck & Leahy, 2002, Dogdas 2005, see brainsuite.usc.edu). These packages can be adapted to integrate with a commercial FEM solver (e.g., SIMPLEWARE LTD. to import MRI scans, segmentation, creating FE mesh, and COMSOL INC. for FEM computation). The result of this integration work is a software package that takes an anatomical MRI and computes the required forward models for each electrode location. Fully automated segmentation is a challenging problem in the art. As such, the accuracy of automated tools are compared to a hand-segmented MRI and evaluated to see if the differences in automated vs hand-segmentation leads to significantly different predicted cortical current distributions.

Variability in anatomy is well characterized by head size, gender, and age.

Therefore, a set of standard forward models will be generated and provided as part of the real-time targeting module.

In an example, the computationally expensive step of computing the FEM is performed in advance for a set of standard electrode locations, while the optimization of the electrode currents to target a specific cortical area is performed in real-time. The GUI allows the experimenter to define the target location and inspect the expected optimal current distribution in real-time.

Simulating Pad-Electrodes with High-Definition Arrays

In another embodiment, an efficient tool to estimate current distributions for arbitrary pad configurations while obviating the need for complex simulation software is provided. Specifically, to efficiently estimate current distributions for arbitrary pad configurations, the pads are simulated with an array of high-definition (HD) electrodes and an efficient linear superposition is used to then quickly evaluate different electrode configurations.

Thus, a method according to this aspect can be performed by data processing apparatus for modeling of transcranial stimulation. The method includes obtaining an image of target tissue, assigning to the image tissue electrical conductance values, arranging a plurality of stimulation electrodes around the target tissue, obtaining, from the locations of electrodes and tissue electrical conductances, a forward model of the response of the tissue to applied currents for each electrode and computing an expected tissue response for an arbitrarily sized and shaped electrode defining an electrode area by combining the forward model for each electrode of an array of electrodes covering the defined electrode area.

In this aspect, each of the plurality of stimulation electrodes is a “small” electrode, such that electrodes do not touch each other, even in a dense array. The use of such “small” electrodes with surface area sizes approaching zero can be implemented on a computer. In fact, as the density of the array increases, the size of the electrodes preferably decreases. This is particularly important in the model where the electrodes should cover only a portion of the surface area. Indeed, it has been found desirable to provide ample open areas between the electrodes. Thus, a large number of point electrodes is the ideal scenario.

The array of electrodes covering the defined electrode area preferably includes at least one high-definition (HD) electrode. The combining of the forward model for each electrode of the array of electrodes covering the defined electrode area involves a linear superposition. Also, the computed expected tissue response preferably includes field intensities or current densities. The forward model can be pre-calculated, wherein the forward model is obtained, for example, from a standard library.

### Example

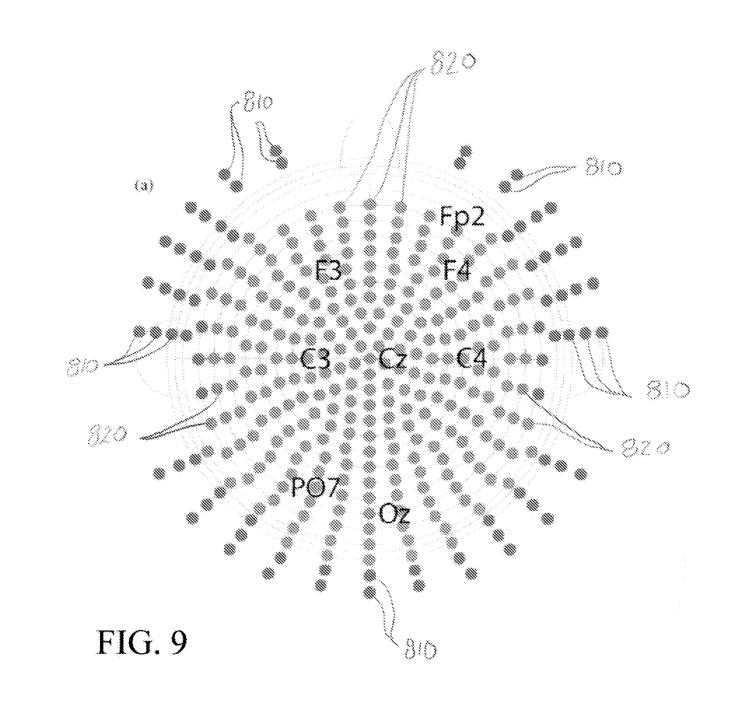

Turning to FIG. 9 showing a two-dimensional schematic of an arrangement of scalp electrodes. Seventy-four outer electrodes 810 were added to the concentric system provided by BioSemi (i.e, 258 inner electrodes 820). Four additional neck electrodes (not shown) were also added. The electrodes designated F3, Fp2, F4, Cz, C3, C4, PO7 and Oz indicate the locations (according to the 10-10-system) of electrodes representing regions of the sampled pad configurations. (b) List of pad configurations used including their sizes and the number of electrodes used to sample these pads.

Numerical results on ten different pad configurations on a normal individual show that electric field intensity simulated with the sampled array deviates from the solutions with pads by only 5% and the locations of peak magnitude fields have a 94% overlap when using a dense array of 336 electrodes. This is significant since computationally intensive FEM modeling of the HD array needs to be performed only once, perhaps on a set of standard heads that can be made available to multiple users. The present results confirm that by using these models one can now quickly and accurately explore and select pad-electrode montages to match a particular clinical need.

A head model with different tissue types was constructed from data by a T1-weighted MRI scan from a healthy subject (male, 36 years old). First, automated segmentation was performed using SPM8 (SPM8, Wellcome Trust Centre for Neuroimaging, London, UK) to demarcate the MRI image into six categories: skin, skull bone, CSF, gray matter (GM), white matter (WM), and air. An in-house MATLAB (Mathworks, Natick, Mass., USA) script was used to correct the automatic segmentation errors. Residual segmentation errors were finally fixed manually in ScanIP (Simpleware, Exeter, UK). Small HD electrodes (4 mm radius to prevent overlap) were placed automatically on 336 locations on the scalp using a MATLAB script. The montage follows a conventional concentric system with additional electrodes. Location coordinates for 258 electrodes were used following the concentric arrangement used by BioSemi (BioSemi B.V., Amsterdam, Netherlands). Additional rows of electrodes were placed to potentially allow stimulation of deeper or lower-lying cortical targets. Four additional electrodes were placed around the neck which may serve to emulate distant reference electrodes. In total this adds up to 336 electrodes (258 in original concentric system+74 in additional rows+4 neck electrodes) (See FIG. 9). After generating the six tissue masks and two masks for electrodes and gel, a complete model of the head can be created.

To model current flow, the head is considered a volume conductor comprised of distinct tissues with a specific and uniform conductivity (assuming isotropic conductivity for simplicity). Current flow is governed by Laplace's Equation, which has an unique solution if the electric field is continuous at tissue boundaries and specified at the outer boundary of the volume. For arbitrary shaped media, no closed form solution exists and one typically uses numerical techniques. Therefore, the volume is discretized into a set of finite elements, each with uniform conductivity, and Laplace's Equation is solved at all discrete elements. Creation and solving of the FEM model followed the procedures described above. ScanIP was used to generate a mesh with tetrahedral elements using adaptive meshing to limit the size of the total model while preserving a 1 mm resolution. The FEM calculation was performed with Abaqus (Simulia, Providence, R.I., USA) using automated batch-processing scripts. The standard Laplace equation was solved using conjugate gradients iterative solver. For each of the 336 HD electrodes the applied current was set to 1 mA by adjusting current-density to the exact area of each electrode. Abaqus solutions on the tetrahedral grid were read back into MATLAB and interpolated on the original 3D regular grid of the MRI segmentation.

For comparison, ten continuous pad configurations were also solved. The pad-electrodes were placed manually on the scalp using ScanIP. The selection of these pad configurations, including varying pad sizes, is based on previous clinical use for the configurations C3-C4, F3-F4, Oz-Cz as well as arbitrary combinations of other locations to quantify the possible variability in the results. Subsequently, these solutions were approximated by sampling the pad with the HD electrodes, i.e. the sampled solutions are a linear superposition of the solutions of the subset of HD electrodes overlapping in location with the pad-electrode. The results are shown in the following table, which lists pad configurations used, including their sizes and the numbers of electrodes used to sample these pads:

(b)

| # | pad configurations | pad sizes | # of electrodes |
|---|---|---|---|
| 1 | Cz-F4 | 5 × 5 - 5 × 5 cm² | 10-10 |
| 2 | C3-C4 | 5 × 5 - 5 × 5 cm² | 9-11 |
| 3 | F3-F4 | 5 × 5 - 5 × 5 cm² | 9-10 |
| 4 | Oz-F4 | 5 × 5 - 5 × 5 cm² | 9-10 |
| 5 | C4-F4 | 5 × 5 - 5 × 5 cm² | 11-10 |
| 6 | F3-C4 | 5 × 5 - 5 × 5 cm² | 10-11 |
| 7 | Oz-Cz | 5 × 7 - 5 × 7 cm² | 9-13 |
| 8 | PO7-F3 | 5 × 5 - 5 × 5 cm² | 12-9 |
| 9 | PO7-F4 | 5 × 5 - 5 × 5 cm² | 12-10 |
| 10 | Oz-Fp2 | 5 × 5 - 5 × 5 cm² | 9-9 |

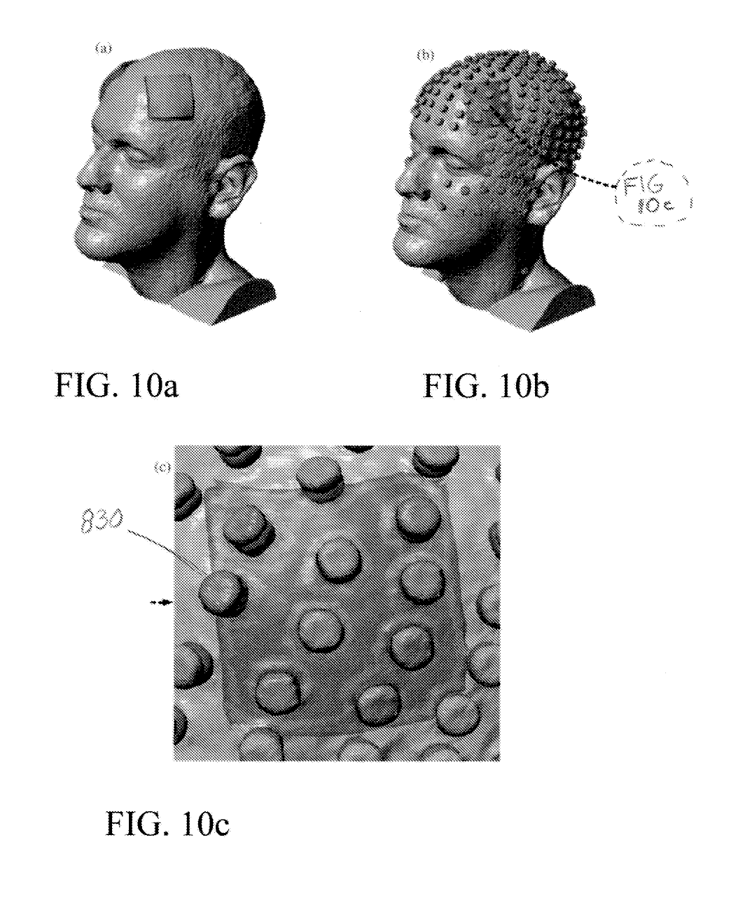

An electrode 830 was included in the sampling subset if at least half of its area was covered by the pad's area, as shown in FIGS. 10a, 19b and 10c. With this criterion and given the fixed locations of the sampling electrodes, a varying number of electrodes resulted for each configuration (See the Table above.) Both, overlap of sampling electrodes as well as their number affects the accuracy of sampling. It is also noted that the size of the pad electrodes for the Oz-Cz configuration differs from the other configurations. With this, the sampling technique for another pad size, (i.e. a rectangular pad of 5×7 cm², instead of only quadratic, 5×5 cm² pads), can be analyzed. The specific choice of pad size was based on previous modeling studies that used those dimensions.

Simulations show that current-density distribution on a continuous pad is not uniform. To explore whether emulating such an uneven distribution on the array could improve performance, three different methods of distributing current among the subset of HD electrodes (uniform, non-uniform, and optimal) were tested.

The simplest approach is a uniform weighting whereby every single electrode is set to the same current intensity value.

In a continuous pad, currents are not uniformly distributed. To emulate this behavior three simplified non-even distributions of currents were tested on the available subset of electrodes. Electrodes were defined as corner, border or center electrodes depending on their distance from the center of the pad (corner: outside of outer ellipse, center: inside the inner ellipse, border: between the two ellipses; the axes of the outer ellipse are given by the pad dimensions and the axes of the inner ellipse are 75% from these values). The following weightings of current-density for corner-border-center were tested: 3-2-2, 6-3-2 and 15-5-2. The specific values were selected to roughly approximate the decay observed in current-density for different pad sizes (larger pads decay faster). The uniform distribution can be represented by the weights 1-1-1.

Since the solutions for the continuous pad configuration are available, a best-case solution can also be computed by optimizing the weightings using least-squares optimization, which are constrained to have correct signs and to add up to a total of zero current (i.e. electrodes in a pad are either cathodal or anodal and all current entering must also exit the head). This can be implemented with linearly constrained least-squares:

sₗₛ=arg min∥E·s−p∥²

subject to sᵢ>0,sⱼ<0 and s^{T}·1=0, (3)

where E is the matrix with the electrode's solutions used to sample the pad, p is the actual pad solution arranged as a vector and s are the weighting factors for each electrode which are to be optimized. i, j indicate the index of the anodal and cathodal electrodes, respectively.

The solutions for the continuous pad and sampled pad were compared in terms of field magnitude and location of peak stimulation, which is perhaps a more relevant criterion for clinical applications. The electric fields obtained for the continuous and sampled pad are denoted by Eₚ and Eₛ, respectively. With these the relative error is defined as:

eᵣₑₗ²=∥Eₚ−Eₛ∥/∥Eₚ∥², (4)

where ∥ ∥² denotes the square sum over voxels and xyz dimensions of the field vector. The calculation of relative errors was performed for gray and WM voxels separately. Areas of peak intensity (upper quartile of field intensities) were compared using the Jaccard index:

J(Pₚ,Pₛ)=|Pₚ∩Pₛ|/|Pₚ∪Pₛ| (5)

Pp, Ps are the peak masks from pad and sampled pad results, respectively. A Jaccard index close to 1 indicates high overlap while an index close to 0 denotes no overlap.

Thus, the linearity of the Laplace equation is leveraged to simulate a single pad-electrode with a linear superposition of smaller electrodes. The comparison of the current distribution between continuous pad and sampled pad reveals the following.

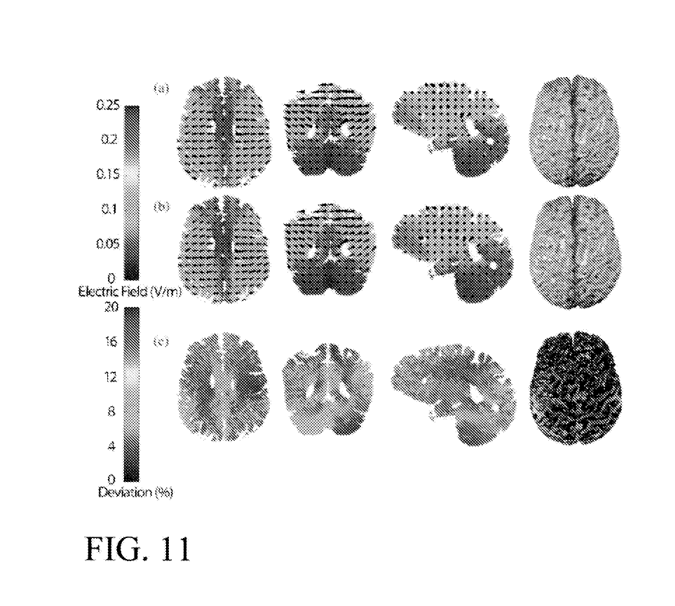

The results for field magnitude on the brain are exemplified for one pad configuration in FIG. 11 showing a good correspondence of the sampled pad with the continuous pad. To quantify the difference, the relative error for all configurations is calculated, using different possible current distribution among the subset of sampling electrodes. With a uniform current distribution the relative error in field magnitude is 5%±0.68% for GM and 4.6%±0.7% for WM (mean±std across ten pad configurations). Other current distributions give similar results with the exception of the optimal solutions, as expected, as it uses additional information.

The relative error calculation for different tissue types, using the uniform distribution among the HD electrodes, reveals an increasing error for tissues that are closer to the electrodes. This is an expected outcome: as one moves further away of the electrodes the blurring of current distributions (primarily at skin/bone and CSF/bone boundaries) reduces the effect of discrete sampling on the surface.

To determine the locations of maximal stimulation and whether they differ significantly for the sampled solution, the Jaccard index (equation (5)) was measured comparing areas of peak field intensity (top 25%). Results indicate a very good overlap with a Jaccard index of 94.3%±2% for GM and WM combined using the uniform distribution.

The Jaccard indices for the different tissue types, also using the uniform distribution, show an increasing overlap of peak intensity locations for tissues that are closer to the electrodes. Again, this is expected as the sampling has been selected precisely to overlap in area, while at a distance the blurring of current flow may lead to a reduced overlap.

The ten different pad configurations are all comparable in terms of errors, which are in the range of 4%-6%. A major determinant for the adequacy of this sampling approach is a complete coverage of the pad with the sampling electrodes.

To investigate the necessary resolution of HD electrodes, the error rates for different number of electrodes used to sample a pad-electrode was computed. Starting with two electrodes (one for each pad) electrodes were incrementally added and the relative error and locations of peak intensity were calculated. Electrodes were selected at random and so this evaluation was repeated 100 times per configuration to obtain a mean behavior with increasing electrode counts. Evidently, increasing the number of HD electrodes leads to a decreasing relative error and an increasing Jaccard index, indicating a higher overlap of peak intensity areas.

To determine if the different methods of distributing currents among the sample electrodes differ, a repeated-measures one-way analysis of variances (ANOVA) was performed with five conditions (uniform distribution, 3-2-2-, 6-3-2-, 15-5-2-weighting and optimal distribution). A significance level of 5% was considered for all hypothesis tests. The test showed a significant difference (p=2.2×10⁻¹⁶) between the various methods. Obviously, the optimal solution outperforms the others, since it uses the pad solution as a prior information. Inspection of these optimal solutions reveals that they are not readily predictable and thus this performance cannot be trivially achieved in practice. Leaving out the optimal solution and conducting an ANOVA for the four remaining methods still results in a significant difference (p=0.0275). A comparison of the mean values shows that the 15-5-2-weighting produces the highest error and leads to this test result. Pairwise t-tests reveal that there is no significant difference between uniform and 3-2-2-weighting (p=0.36), 6-3-2-weighting (p=0.85) and 15-5-2-weighting (p=0.17). Comparable results were obtained for the WM tissue. In summary, none of the tested weight-distributions outperform the naive uniform distribution.

Thus, it has been shown that sampling provides a fast approach to simulate the current distribution in the head for the use of tES, with a relatively small decrement in accuracy. Sampling is sufficient due to the significant amount of blurring on the head surface and CSF. The focal spot of an HD electrode is broadly distributed at the level of cortex due to lateral current flow on skin and CSF. To be specific, for a spherical head model one can show that a point source on the skin surface results in a focal spot of approximately 27° in width at the cortical surface. Thus, focal spots generated by two small HD electrodes that are 9° apart are already no longer resolvable at the cortical surface.

The uniform distribution over all electrodes leads to the most robust results with an error of no more than 6%. A corresponding graphical user interface that uses this sampling approach is forthcoming based on existing commercial software (HDExplore, Soterix Medical Inc., used for instance in).

Computer Components

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

All references and publications disclosed or discussed herein are incorporated by reference in their entirety.

1. A method performed by data processing apparatus for modeling of transcranial stimulation, the method comprising:
obtaining an image of target tissue;
assigning to the image tissue electrical conductance values;
arranging a plurality of stimulation electrodes around the target tissue;
obtaining, from the locations of electrodes and tissue electrical conductances, a forward model of the response of the tissue to applied currents for each electrode; and
computing an expected tissue response for an arbitrarily sized and shaped electrode defining an electrode area by combining the forward model for each electrode of an array of electrodes covering the defined electrode area.

2. The method of claim 1, wherein each of the plurality of stimulation electrodes is small enough so as to not touch each other.

3. The method of claim 1, wherein the array of electrodes covering the defined electrode area includes at least one high-definition (HD) electrode.

4. The method of claim 1, wherein the combining of the forward model for each electrode of the array of electrodes covering the defined electrode area comprises a linear superposition.

5. The method of claim 1, wherein the computed expected tissue response includes field intensities or current densities.

6. The method of claim 1, wherein the forward model is pre-calculated.

7. The method of claim 6, wherein the forward model is obtained from a standard library.

8. The method of claim 1, wherein the electrical stimulation applied is the same for each electrode in the plurality of electrodes.

9. The method of claim 1, wherein the image is derived from a pre-existing image library or a target tissue specific image.

10. The method of claim 1, wherein the forward model is computed using an finite-element model of a boundary element model of the tissue properties.

11. The method of claim 1, wherein the electrical conductance are non-isotropic and/or non-uniform.

12. The method of claim 1 wherein the image is derived from a fluoroscopic image, an MRI image, a CT image, or a combination of imaging techniques.

13. The method of claim 1, wherein the plurality of electrodes are placed around the target tissue based on anatomical landmarks.

14. The method of claim 1, wherein the plurality of electrodes are placed around the target tissue in a pattern on the skin, below the skin or within the target tissue.

15. The method of claim 1, wherein the electrical stimulation applied is different for each electrode in the plurality of electrodes.

16. The method of claim 1, wherein the obtained image of target tissue comprises a standard library image, a target tissue specific image, a fluoroscopy image, an MRI image, a CT image or a combination of images.

17. The method of claim 1, wherein the forward model is calculated utilizing a processor or processors in communication with a storage medium.
